(19) United States
(12) Patent Application Publication
(10) Pub. No.: US 2007/0123865 A1
(43) Pub. Date: May 31, 2007

(54) Device for the dynamic stabilization of bones

(76) Inventors: Fridolin Schlapfer, Holstein (CH); Martin Hess, Holstein (CH)

Correspondence Address: JONES DAY, 222 EAST 41ST STREET, NEW YORK, NY, 10017-6702, US

(21) Appl. No.: 11/588,623

(22) Filed: Oct. 26, 2006

Related U.S. Application Data
(63) Continuation of application No. PCT/CH04/00257, filed on Apr. 28, 2004.

(51) Int. Cl. A61F 2/30

(52) U.S. Cl. 606/61

(57) ABSTRACT

A device for the dynamic stabilization of bones or bone fragments, especially vertebral bodies, includes a longitudinal carrier having a longitudinal axis and at least two bone anchoring devices. Each bone anchoring device has an anchoring part that attaches to bone and a connecting part that detachably fastens the longitudinal carrier to the anchoring device. The longitudinal carrier has in the axial direction a number of n flexible segments where n≧1. Each flexible segment has a cross-sectional surface orthogonal to the longitudinal axis that corresponds neither to a circle nor to a regular polygon. The longitudinal carrier also has a number of m essentially stiff segments where m=(n+1). The total number of flexible and stiff axial segments is ≧(2n+1).

### CROSS-REFERENCE TO RELATED APPLICATION

This is a continuation of pending International Application No. PCT/CH2004/000257, filed Apr. 28, 2004, the entire contents of which are expressly incorporated herein by reference thereto.

### FIELD OF THE INVENTION

The invention relates to a device for the dynamic stabilization of bones and bone fragments, especially of vertebral bodies.

### BACKGROUND OF THE INVENTION

The spinal column is the center of a complex process for counterbalancing external and internal forces and moments. The forces and moments are balanced by muscles, with the spinal column as abutment.

At the same time, the spinal column has a form-giving function (bending forwards and backwards, dislocating, etc.) and also plays an important role in damping.

When stabilizing segments of the spinal column affected by degenerative diseases, fractures, deformities, etc., care must be taken to ensure that the effect of the stabilization on the functions of the spinal column is kept to a minimum.

In cases where corrective forces are not necessary, the affected segments are not fused. Instead, weakened structures are selectively supported internally by a dynamic system connected with the spinal column. In this regard, realize that, in contrast to intervertebral disk prostheses, the structures in question are not removed. Accordingly, depending on the degree of degeneration, a restoration of the physiological movement pattern does not necessarily solve the clinical problem.

The main indications for a dynamic system are diseases, inflammations and/or injuries in the region of the intervertebral disk, the ligamentous apparatus, the faceted joints and/or subchondral bones. In these situations, it is important that
a) the pain is alleviated and the progress of the pathology is, at the very least, frozen in place and
b) a residual mobility, supporting the metabolism of the structures (intervertebral disks, ligaments, faceted joints) remains.

Looking at them from the posterior to the anterior, the faceted joints, the posterior annulus, and the subchondral bones below the bony upper plate of the vertebral body are clinically identifiable pain zones. The pain may be strictly mechanical in nature or initiated by aseptic inflammations (by mechanical and/or chemical irritations). Amelioration of the pain and healing of the inflammations require a reduction in the load and, with that, the extension of the structures in question.

The AO spinal column external fixator (description of the system in “Fixateur externe”, authored by B. G. Weber and F. Magerl, Springer-Verlag 1985, pages 290-366) is used at the present time primarily for diagnostic purposes. Because of its configuration, it is very stiff in shear and rotation and flexible in flexion/extension and inclination to the side. Due to the extreme posterior position of the frame of the external fixator, the intervertebral disk and the bony upper plate of the adjoining vertebrae are stressed more or less uniformly in compression during flexion and extension. Contrary to this, an uncoiliing over the central nucleus takes place when the movement segment is intact. At the same time, the posterior and anterior parts of the annulus are stressed alternatively in tension and compression. The greatest load on the bony upper plate of the adjoining vertebrae occurs in the region of the nucleus. Since the nucleus material represents a gelatinous composition, the latter is worked back and forth during flexion and extension and, at the same time, pressed against the annulus. If the annulus has cracks because of a degenerative process or a traumatic incident, nucleus material penetrates into the cracks during in this working movement. If, in so doing, the nucleus material penetrates into the innervated and vascularized outer region of the annulus or even from the intervertebral disk and comes into contact with the corresponding nerve roots, inflammation processes with the corresponding pain may be initiated. The movement pattern resulting from the interaction between the external fixator and the intervertebral disk, and consisting of a more or less vertical movement in the region of the intervertebral disk reduces the risk that nucleus material is pressed or pumped into the outer region of the annulus.

With the exclusion of the working movement during flexion and extension, the stress on the bony upper plate of the adjoining vertebrae and, with that, the stress on the subchondral bone underneath is relieved simultaneously by the external fixator. In the case of inflammatory changes in the subchondral bone (described by modic change 1), this stress relief may lead to a reduction in pain. With the release of stress on the upper plates, prerequisites are also created for the healing of the inflammation.

If the nucleus is dehydrated, there is no radial pressure on the annulus, with the result that high local stresses on the annulus, resulting during flexion and extension, can lead to the delamination of the annulus. As already described above, the pressure arising during flexion and extension is distributed uniformly over the whole of the intervertebral disk by the external fixator. With this, the risk of a delamination of the posterior annulus is reduced.

Clinical experience with the diagnostically used AO spinal column external fixator shows that a system, which is stiff in rotation and shear and flexible in flexion/extension and side inclination, can lead to a reduction in the symptoms.

Since the AO spinal column external fixator is used only for diagnostic purposes and, with that, only briefly, the clinical experience gained with the system provides no information of the extent to which the metabolism of the soft tissue structures is maintained with a system such as the external fixator.

The system, described in the EP 0 669 109 B1 patent (Baumgartner) has successfully been used clinically for some years. Due to the posterior position of the longitudinal carrier, the situation that arises in the intervertebral disk region is similar to that which arises in the case of the AO spinal column external fixator. Many years of clinical experience with the system indicates that the metabolism of the soft tissue structures is maintained.

Based on the clinical experience with the AO spinal column internal fixator and the system described in the EP 0 669 109 B1 patent (Baumgartner), a system used to support spinal column structures should be stiff in rotation and shear and flexible in flexion and extension, in order, on the one hand, to reduce the symptoms and, on the other, to maintain the metabolism of the structures affected, the latter, in order to create the prerequisites for biological remodeling and healing. Such a system typically includes a longitudinal carrier and at least two bone anchoring devices.

The system, described in the EP 0 669 109 B1 patent (Baumgartner) displaces the point of rotation of the spinal column segment from the intervertebral disk in the posterior direction into the region of the faceted joints. In comparison to the AO spinal column external fixator, the system exhibits reduced stiffness in rotation and shear because of the centrally located ligament.

Aside from the reduced stiffness in rotation and shear, the EP 0 669 109 B1 patent (Baumgartner) also has the following disadvantages:
expensive threading of the supporting element on the ligament;
cutting the supporting elements to the desired length—in so doing, care should be taken to ensure that the cut surfaces will be parallel to the side surfaces of the bone screws; and.
if the bone screws are introduced diverging to the front, it is difficult to bring the supporting elements between the screw heads from the rear.
   Due to the central position of the ligament, the system does not automatically spring back into the starting position.

Ball joint-like connections between the pedicle screw and the longitudinal carrier, as described, for example, in patent documents WO 94/00066 (Schlapfer) and PCT/CH02/00672 (Schläpfer) permit the implant to be installed stress-free with subsequent locking of all degrees of freedom.

In order to ensure a certain dynamic behavior of the construct in spite of the angularly stable connection between the pedicle screws and the longitudinal carrier, either the two upper (cranial) ball connections between the upper pedicle screws and the longitudinal carriers can be left loose or the longitudinal carriers themselves can be configured flexibly.

Flexible longitudinal carriers are described, for instance, in patent documents DE 42 39 716 C1 (Winter), WO 95/27444 (Alby), WO 98/22033 (Elberg), WO 02/102259 (Sengupta) and WO 93/20771 (Mazel). The two first-mentioned documents comprise systems that include movable parts. Movable parts have the major disadvantage of attrition.

The two documents WO 98/22033 (Elberg) and WO 02/102259 A2 (Sengupta) describe systems with bent, springy longitudinal carriers. In the case of the first, the longitudinal carrier has a stop, which limits the bending in one direction. Properly designed, the systems described in these two documents enable the weakened structures of the spine to be stabilized dynamically.

WO 93/20771 (Mazel) describes a system that includes flexible rods. Here also, if designed correctly, dynamic stabilization of weakened structures of the spine is possible.

### SUMMARY OF THE INVENTION

It is an object of the invention to develop a longitudinal carrier, which can be connected to bone anchoring devices and has maximum flexibility in bending at a specified strength, especially a shearing stiffness.

Pursuant to the invention, this objective is accomplished with a device for the dynamic stabilization of bones and bone fragments, especially of vertebral bodies, having a longitudinal carrier comprising a number of flexible segments and a number of stiff segments in the axial direction of the carrier.

The advantages of the invention in comparison to the existing systems include the following:
maximum bending flexibility at a specified strength;
decoupling sheering stiffness from bending flexibility; and
a combination with the three dimensional connections between longitudinal carriers and bone anchoring means, described in WO 94/00066 (Schläpfer) and PCT/CH 02/00672 (Schläpfer), enables the longitudinal carrier to be installed stress free.

When a fusion is carried out, the system, used for the stabilization should have a sufficiently large stiffness so that the movement in the movement segment, which is to be fused, is so small that a bony bridging of the vertebrae takes place. Mechanically, the system used for the stabilization is connected parallel to the spinal column segment, which is to be fused, that is, the stiffer the system, the larger is the proportion of the spinal column load that is passed over it. The system should be designed correspondingly strong.

In contrast, as explained in detail above, bending flexibility in combination with compression, rotation and shearing stiffness is required for dynamic stabilization of a spinal column segment.

For the mechanical dimensioning, a dynamic fixation system with said properties should be dimensioned for a maximum deformation in relation to bending and for a maximum load in relation to compression, shear and rotation. The corresponding conditions can be approximated with the linear elasticity theory.

In the case of a dynamic fixation system, anchored from the posterior direction by the pedicle, the longitudinal carriers are stressed predominately in bending. Correspondingly, the conditions explained above can be described for the individual longitudinal carriers, which act as cantilever beams, by
a) The differential equation of the elastic bending line of the beam axis (Eulers elastic deformations)

$$\frac{{w ( z )}^{″}}{{( 1 + {w ( z )}^{′2} )}^{3 / 2}} = \frac{{M}_{b} ( z )}{E * {l}_{y} ( z )} \qquad ( 1 )$$
b) The moments equations
   M_{b}(z)=M_{y}+F_{y}*(I−z)−F_{z}*w(z) (2)
c) Hook's Law

$${σ}_{b} ( x , z ) = \frac{{M}_{b} ( z )}{{l}_{y} ( z )} * z \qquad ( 3 )$$
d) and the Maxwell (von Mises) or Tresca flow conditions
   σₓ²+σ_{z}²−σₓ*σ_{z}+3*τ_{xz}² (x,z) von Mises (4a)
   σₓ²+σ_{z}²−σₓ*σ_{z}+4*τ_{xz}² (x,z) Tresca (4b)

By varying the thickness of the cross section and/or the material stiffness (with or without additional fiber reinforcement, such as carbon fibers) along the longitudinal carrier, it becomes possible to combine bending flexibility with shearing stiffness. The shear forces are transferred over the cranial pedicle screws to the construct [symbol] the bending load, brought about by the shear forces in the construct, increases linearly from the cranial (0) to the caudal (maximum). In contrast to this, bending moments acting in the sagittal plane bring about a homogeneous bending load over the whole length of the construct. If now the longitudinal carrier of the construct is configured as stiffly as possible caudally and as flexibly as possible cranially, the construct is able to absorb shear forces and still bend flexibly.

The individual longitudinal carriers consists of “z” segments disposed serially, z≧3. The transition between the individual segments may be continuous or discrete (abrupt).

The individual segments differ in the size and shape of the cross section and/or the material stiffness. The cross section, as well as the material stiffness may vary within the segments.

Of the z segments, at least the end segments are at least twice as stiff in bending as the “n” flexible segments between the end segments.

As already mentioned, the “n” flexible segments with 1≦n≦z−2 are designed so that they do not fail mechanically at a specified bending deformation of the longitudinal carrier. Said bending deformation of the longitudinal carrier is the sum of the individual deformations arising in the individual flexible segments.

The cross-sectional surfaces of the n flexible segments may, for example, be rectangular, trapezoidal, rhomboid, triangular (but not equilateral), oval or elliptical.

In a preferred embodiment, the cross-sectional area of at least one of the n flexible segments is constructed rectangularly with a flattened cross-sectional surface. The advantage of this configuration lies in that, at a given flexibility, an optimum strength is achieved with a rectangular configuration of the cross section of the flexible segments of the longitudinal carrier. The bending flexibility may be combined with the rotational stiffness and shearing stiffness by varying the cross section from caudal to cranial (caudal thick, cranial thin). The maximum flexibility in bending, which is the aim at a specified strength, can be attained only by a rectangular configuration of the cross section of the flexible part of the longitudinal carrier. A rectangular configuration of the longitudinal carrier requires a three-dimensional, spherical connection between the longitudinal carrier and the pedicle screw, as described, for example, in WO 94/00066 (Schläpfer) and PCT/CH02/00672 (Schläpfer).

The z−n essentially stiff segments of the longitudinal carrier preferably are cylindrical or prismatic, their cross-sectional area orthogonal to the longitudinal axis and preferably configured radially symmetrically. Axially on the outside, the longitudinal carrier comprises one of these z−n stiff segments, so that the longitudinal carrier can be connected by means of these outer segments in the connecting parts at different—in the case of a circularly cylindrical configuration of these outer stiff segments, at any—angle of rotation relative to the longitudinal axis. The cross-sectional area of these z−n stiff segments, especially of the outer stiff segments, preferably is circular.

On the assumption that the cross section and the material stiffness of the segment remain constant over its length L, the following apply for n=1 flexible segments:
Thickness≦σ_{zul}/E×“free length”×2/tg α
e≦σ_{zul}/E×1×28 for α=4°
wherein:
α is the deformation angle of the longitudinal carrier, between 4° and 12° and preferably 8°
e is the thickness of an elastic segment, measured perpendicularly to the bending axis
L is the length of the elastic segment
σ_{zul} is the dynamic bending strength of the material (such as 48 N/mm² for plastics up to 1,400 N/mm² for steel)
E is the modulus of elasticity (28 N/mm² for plastic up to 220,000 N/mm² for steel)
   σ_{zul}/E 0.5≧σ_{zul}/E≧0.005

Preferably, the n flexible segments are configured with dimensions within the following limits:
width b between 8 mm and 15 mm, preferably between 10 mm and 13 mm;
thickness e between 0.5 mm and 3 mm, preferably between 0.9 and 1.1 mm; and
length L between 2 mm and 30 mm, preferably between 5 mm and 15 mm.

For n>1 flexible segments, the following applies:
eⁱ≦σⁱ_{zul}/Eⁱ×Lⁱ×2/tg αⁱ
with i=1 to n
Σ^{i=1 bis n}α¹=α 4°≦α≦12°

Preferably, the n flexible segments are configured with dimensions within the following limits:
width b between 8 mm and 50 mm, preferably between 10 mm and 13 mm;
thickness e between 0.5 mm and 3 mm, preferably between 0.9 and 1.1 mm; and
length L between 2 mm and 30 mm, preferably between 5 mm and 15 mm.

The longitudinal carrier preferably is produced from one of the following materials:
high-strength titanium;
high-strength titanium alloy;
high-strength steel;
a cobalt-chromium alloy;
a high-strength, monocrystalline material; or
a high-strength monocrystalline fiber-reinforced material.

### BRIEF DESCRIPTION OF THE DRAWINGS

The invention and further developments of the invention are explained in even greater detail by means of partially diagrammatic representations of several examples, in which:

FIG. 1 shows a perspective view of an embodiment of the invention;

FIG. 2 shows a cross-sectional surface, orthogonal to the longitudinal axis, of a flexible segment of the longitudinal carrier in one embodiment of the inventive device;

FIG. 3 shows a section through a bone anchoring means of an embodiment of the inventive device;

FIG. 4 shows a view of an embodiment of the invention as a spinal column stabilizing device;

FIG. 5a shows a diagrammatic representation of a longitudinal carrier of an embodiment of the inventive device, attached by means of two bone anchoring means to two adjacent vertebral bodies;

FIG. 5b shows a section of a flexible segment of a longitudinal carrier of an embodiment of the inventive device; and

FIG. 6 shows a diagram for the simplified representation of the break criteria for the longitudinal carrier of an embodiment of the inventive device.

### DETAILED DESCRIPTION OF THE INVENTION

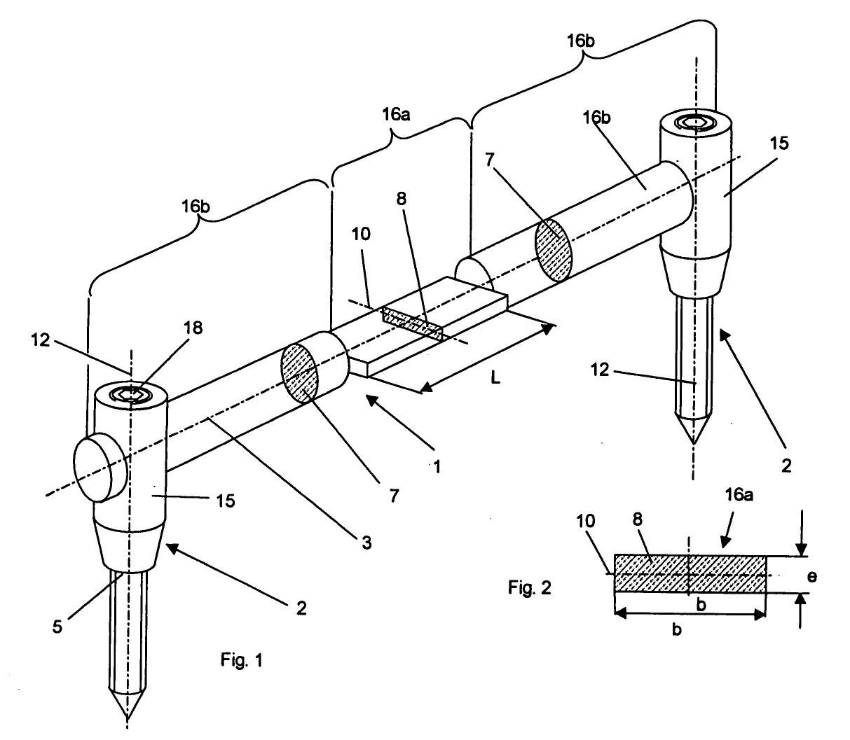

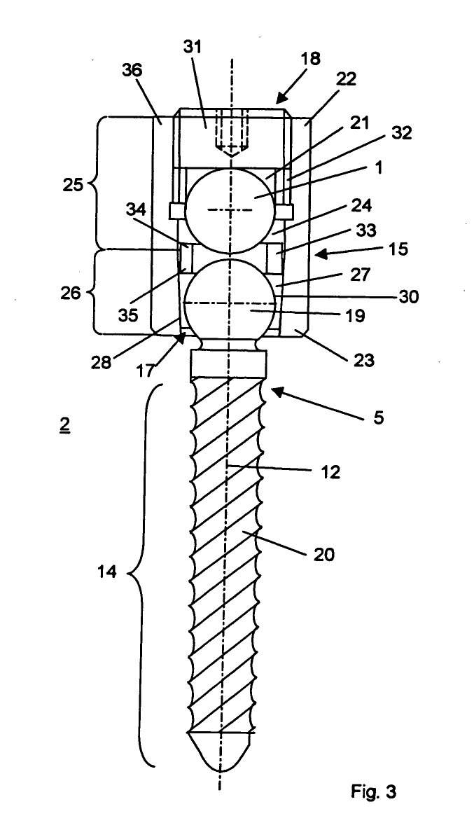

FIG. 1 shows an embodiment of the invention that includes a longitudinal carrier 1 with a longitudinal axis 3 and two bone anchoring means 2, which are constructed as pedicle screws 5 with integrated connecting parts 15 and respective central axes 12. The longitudinal carrier 1 has a total of z=3 segments 16, the two axially outside segments 16b being stiff and the one flexible segment 16a (n=1) having a length L disposed between the two stiff segments 16b. As shown in FIG. 2, the flexible segment 16a, which is disposed in the middle, has a rectangular cross sectional area 8, which is orthogonal to the longitudinal axis 3 with a width b and a thickness e. Width b of the rectangular cross-sectional area 8 is disposed transversely to the longitudinal axis 3 of the longitudinal carrier 1 and transversely to the central axes 12 of the bone anchoring means 2. Due to its shape, the flexible segments 16a can be bent elastically preferably with respect to a bending axis 10 coinciding with or parallel to the long axis of the rectangular cross-sectional surface 8. Over connecting parts 15, the bone anchoring means 2 are detachably connected with the stiff, circularly cylindrical segments 16b of the longitudinal carrier 1.

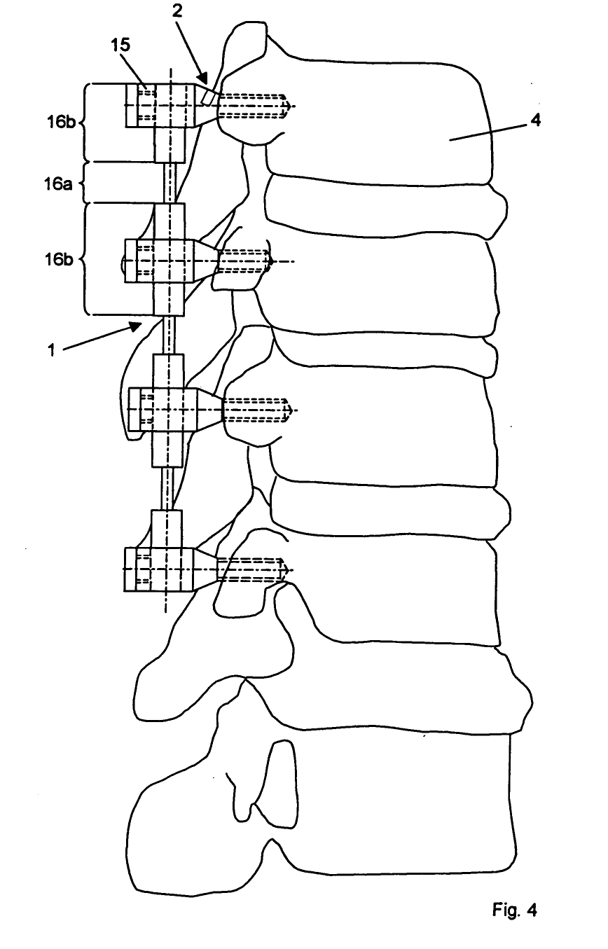

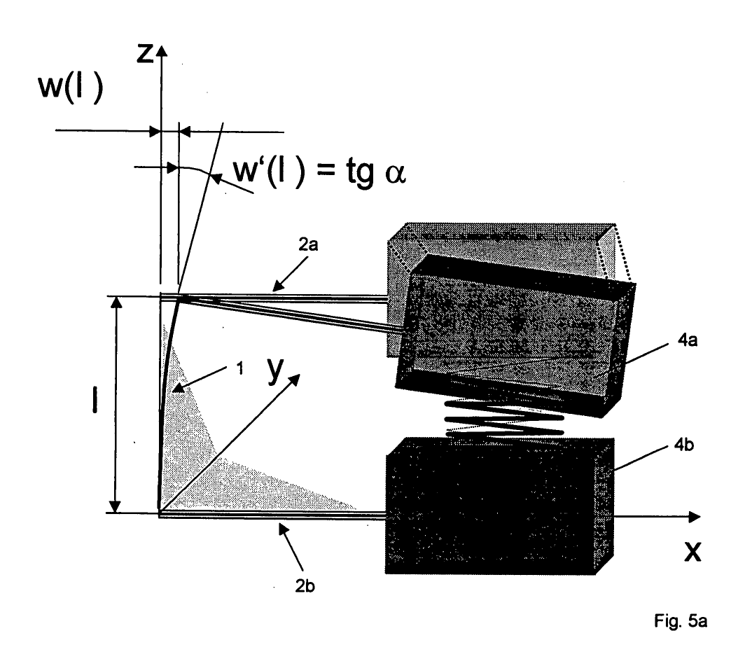

The embodiment of the bone anchoring means 2, shown in FIG. 3, comprises pedicle screws 5, each of which comprises a spherical screw head 19 and an anchoring part 14, which can be screwed into a pedicle of a vertebral body 4 (FIG. 4). The spherical screw head 19 forms one component of the ball joint 17, by means of which the bone anchoring means 2 can be connected polyaxially pivotably with the connecting part 15.

These pedicle screws 5 consist of a coaxial screw shaft 20 and a spherical screw head 19 adjoining the screw shaft at the top and disposed concentrically. The connecting parts 15 are constructed so that the longitudinal carrier 1, before it is fixed to the bone anchoring means 2, can be placed in a channel 21 disposed in a hollow body 36 and subsequently fixed in the channel 21 by clamping means 18. The channel 21 passes through the hollow body 36 transversely to the central axis 12 and is open at the upper end 22 of the hollow body 36.

The hollow body 36 comprises an upper end 22 intersecting the central axis 12, a lower end 23 intersecting the central axis 12, and a cavity 24 that passes through the hollow body 36 coaxially from the upper and 22 to the lower end 23. The cavity 24 has two segments 25,26, which are disposed axially behind one another. The upper segment 25 comprises a coaxial cylindrical borehole in which the radially elastically deformable clamping element 27 is mounted so that it can be shifted axially. The lower segment 26 is constructed so as to taper conically towards the lower end 23 of the connecting part 15. The outer wall 28 of the clamping element 27 is configured complementarily conically to the inner cone 29 in the lower segment 26, so that the clamping element 27 is compressed radially when the cavity 24 is pressed coaxially against the lower end 26 of the connecting part 15. Furthermore, the clamping element 27 comprises an axially continuous open cavity 30, which is constructed complementarily spherically to the screw head 19. In the decompressed state of the clamping element 27, the screw head 19 can be snapped from the lower end 26 of the connecting part 15 into the cavity 30. In the compressed state of the clamping element 27, the screw head 19 is locked in the cavity 30. Because of the spherical configuration of the screw head 19 and of the cavity 30, the bone anchoring means 2 can be swiveled polyaxially to the connecting part 15 and can also be locked at different angles between the central axis 12 of the bone anchoring means 2 and the axis of the connecting part 15.

The clamping element 27 is shifted axially by clamping means 18, which is constructed as a locking screw 31 and can be screwed into an internal thread 32, which is complementary to its thread in the upper segment 25 of the cavity 24. When tightened, the front end of the locking screw 31 presses on the longitudinal carrier 1, which has been placed in the channel 21. An annular adapter 33 is disposed between the longitudinal carrier 1 and the clamping element 27 so that the screw head 19, as well as the longitudinal carrier 1, can be fixed in the connecting parts 15 when the clamping means 18 is tightened. The depth T of the channel 21 is of such a size that the longitudinal carrier 1, when placed in the channel 21, presses on the upper end 34 of the adapter 33. The lower end 35 of the adapter 33 rests on the clamping element 27. When the clamping means 18 is tightened, it presses on the longitudinal carrier 1, so that the adapter 33, together with the clamping element 27 adjoining channel 21, is pressed against the lower end 23 of the connecting element 15. The conical clamping element 27 is compressed radially by the lower segment 26 of the cavity 24, which is constructed conically on the inside, and the screw head 19 is locked in the cavity 30 of the clamping element 27.

FIG. 4 shows a use of the longitudinal carrier 1 within a spinal column stabilizing device. The longitudinal carrier 1 has z=7 segments 16, of which n=3 flexible segments 16a and z−n=4 are stiff segments 16b. Each stiff segment 16b is connected with the connecting part 15 of a bone anchoring means 2. Moreover, in each case, one bone anchoring means 2 is screwed into a pedicle of the vertebral body 4.

Description of the implantation procedure:
screw in pedicle screws with polyaxial heads;
determine distance between the pedicle screws;
select longitudinal carrier (pre-bent) according to the intervals measured;
insert longitudinal carrier;
close polyaxial heads of the pedicle screws;
compress or extend pedicle screws as required; and
lock connection between pedicle screw and longitudinal carrier.

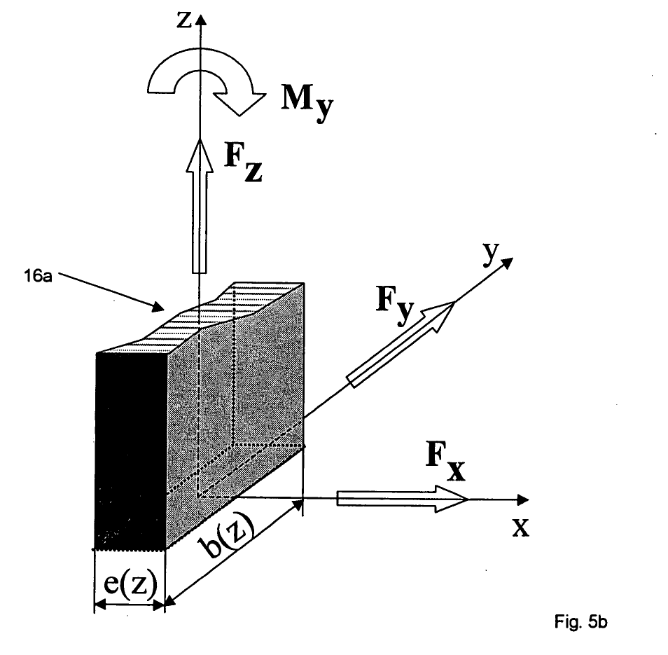

FIGS. 5a and 5b illustrate the relationship between the deformation of the spinal column segment in question and the geometry of the longitudinal carrier, as described in the following block diagram. 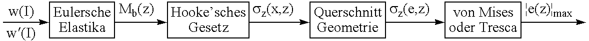
Key for above Diagram:
Eulersche Elastika=Eulers elastic deformation
Hooke'sches Gesetz=Hooke's Law
Querschnitt Geometrie=Geometry of the cross section
oder=or

e(z)ₘₐₓ represents the maximum possible thickness of the longitudinal carrier, so that fracture of the longitudinal carrier does not occur during cranial movement produced by the deformation of the longitudinal carrier.

w(I) and w′(I)=tg α are known from functional x-ray images and/or from in vitro tests.

Strictly speaking, since w(I) and w′(I) depend on the stiffness of the dynamic fixation system and the bridged spinal column segment, ¦e(z)¦ₘₐₓ can be determined only iteratively. However, if it can be assumed that the bending stiffness of the bridged spinal column segments is much smaller than the bending stiffness of the bridged spinal column segments (which is the case), w(I) and w′(I) can be measured directly from the functional x-ray images of patients, who were taken care of with a functionally similar fixation system.

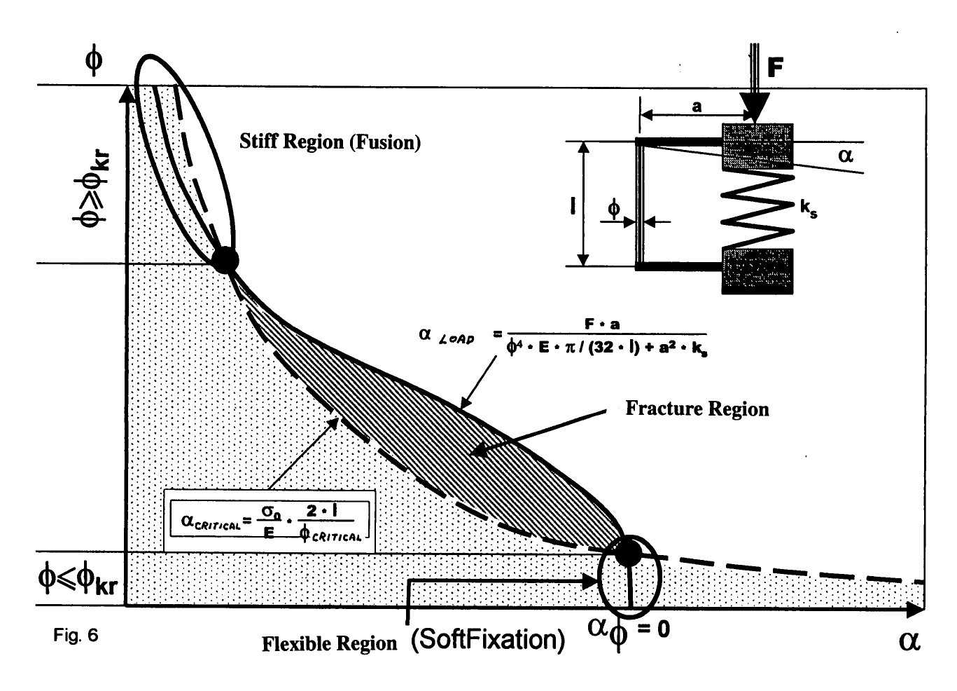

To summarize, it may be stated that the objective of SoftFixation no longer is a fusion, but a functional support of the structures of the bridging spinal column segments. In this regard, the fixation system should be mechanically yielding to some extent. Accordingly, in contrast to a stabilization working towards a fusion, the SoftFixation system is dimensioned for maximum deformation and not for maximum load. However, as shown in FIG. 6, a flexible system is break-resistant only from a certain flexibility onward, that is, the system must be dimensioned so that the flexibility of the fixation system does not fall below the critical value at a given deformation.

The graph shown in FIG. 6 is a simplified representation of the fracture criteria for a fixation system (shown symbolically at the top right) to stabilize a spinal column segment. The following assumptions are made: the spinal/pedicle screw interface and pedicle screws are rigid; the intervertebral disk exhibits a linear elastic behavior with a stiffness kₐ; and two longitudinal carriers have a diameter φ.

The α_{critical} curve shows a simplification of the relationship between the geometry of the fixation system (symbolized by the diameter Φ of the longitudinal carrier) and the maximum deformation (symbolized by the deformation angle α_{critical}), which can be compensated for by the system, before the latter breaks.

The curve also shows that the critical region is shifted to the right as the strength and flexibility of the material increase.

The α_{Load} graph is the characteristic curve of the fixation system. It shows the relationship between the geometry (symbolized by the diameter φ of the longitudinal carrier) and the deformation of the fixation system (symbolized by the deformation angle α) for a specified spinal column load (symbolized by F.) as a function of the mechanical properties of the fixation system (symbolized by the tensile strength σ_{D} and the modulus of elasticity E) and a spinal column segment (symbolized by the linear elastic spring constant kₛ).

As long as the characteristic line α_{Load} of the system is to the left of the α_{Load} graph, there is no danger that the fixation system will break.

In FIG. 6, the curves intersect twice. For the dimensioning of a fixation system, the region above the upper intersections of the two curves was used.

The objective of the stabilization with a fixation system was the fusion of the bridged segments. As the graph shows, the stiffness of the system (given, for instance, by the diameter of the longitudinal carrier) should not fall below a certain value.

In the case of SoftFixation, the objective no longer is a fusion but a functional support of the structures of the bridged spinal column segments. In this regard, the fixation system should be mechanically yielding to some extent. Accordingly, in contrast to a stabilization acting towards a fusion, the fixation system of SoftFixation should be dimensioned for a maximum deformation and not for a maximum load. As the present graphs show, a flexible system is break resistant only from a certain flexibility onward, that is, the system should be dimensioned so that the flexibility of the fixation system does not fall below the critical value for a given deformation. The invention relates to the definition of this condition and to its various realization possibilities.

We claim:

1. A device for the dynamic stabilization of bones or bone fragments comprising:
a longitudinal carrier having a longitudinal axis and comprising in the axial direction:
a number of n flexible segments where n is ≧1, each having a cross-sectional surface orthogonal to the longitudinal axis that corresponds neither to a circle nor to an equilateral polygon,
a number of m substantially stiff segments where m=(n+1), and
a total number z of flexible and stiff segments where z is ≧(2n+1); and
at least two bone anchoring devices each having a central axis, a connecting part, and an anchoring part that attaches to bone, each bone anchoring device detachably fastenable to the longitudinal carrier via the connecting part.

2. The device of claim 1 wherein at least one of the flexible segments has a constant cross-sectional area over the whole of its length.

3. The device of claim 1 wherein the cross-sectional area changes along the longitudinal axis in at least one of the flexible segments.

4. The device of claim 3 wherein the cross-sectional area changes continuously along the longitudinal axis.

5. The device of claim 1 wherein the modulus of elasticity of the material of at least one of the flexible segments is constant over the whole length of the at least one flexible segment.

6. The device of claim 1 wherein the modulus of elasticity of the material of at least one of the flexible segments changes along the longitudinal axis.

7. The device of claim 6 wherein the modulus of elasticity of the material changes continuously along the axis.

8. The device of claim 1 wherein the thickness eᵢ of the flexible segments is so small that:
the sum of the deformations α^{I} of the individual flexible segments gives a total deformation α of the longitudinal carrier between 4° and 12°; and
the maximum bending stresses occurring in the segments do not exceed the permissible bending stresses of the material.

9. The device of claim 8 wherein the longitudinal carrier has a flexible segment with a cross-sectional area and modulus of elasticity that remain constant over the whole length of the flexible segment.

10. The device of claim 9 wherein the ratio of the product of the “thickness e of the flexible segment multiplied by the modulus of elasticity in bending of the material” and the “length L of the flexible segment multiplied by the permissible bending stress of the material” is between 9 and 28.

11. The device of claim 1 wherein the cross-sectional area of at least one of the n flexible segments is rectangular.

12. The device of claim 1 wherein the longitudinal carrier has at each axial end a stiff segment with a cross-sectional area that is radially symmetrical and orthogonal to the longitudinal axis.

13. The device of claim 1 wherein the longitudinal carrier has m=(z−n) substantially stiff segments with a cross-sectional area that is radially symmetrical and orthogonal to the longitudinal axis.

14. The device of claim 13 wherein the n flexible segments and the m substantially stiff segments alternate with one another.

15. The device of claim 1 wherein at least one of the n flexible segments has a cross-sectional surface with a width b and a thickness e, the ratio of e:b being at most between 0.3 and 0.4.

16. The device of claim 1 wherein at least one of the n flexible segments has a cross-sectional surface with a width b and a thickness e, the ratio of e:b being at least between 0.05 and 0.1.

17. The device of claim 1 wherein the longitudinal carrier is one piece.

18. The device of claim 1 wherein at least one of the n flexible segments is connectable to an adjoining stiff segment.

19. The device of claim 1 wherein at least one of the n flexible segments is made from a biocompatible plastic.

20. The device of claim 1 wherein at least one of the n flexible segments comprises a high strength, monocrystalline material.

21. The device of claim 1 wherein at least one of the n flexible segments is reinforced by high strength, monocrystalline fibers.

22. The device of claim 1 wherein at least one of the n flexible segments comprises a fiber-reinforced, biocompatible plastic.

23. The device of claim 1 wherein at least one connecting part comprises a ball joint for the polyaxial, pivotable connection of the bone anchoring device to the longitudinal carrier.

24. The device of claim 1 wherein a cross-sectional area of at least one of the n flexible segments is oval.

25. The device of claim 1 wherein at least one of the n flexible segments is hollow.

26. The device of claim 25 wherein at least one of the n flexible segments has an axially extending borehole.

27. The device of claim 1 wherein each of the n flexible segments axially has a first and a second end, each of which adjoins an essentially stiff segment and that the cross-sectional surface of at least one of the n flexible segments increases in at least one dimension in the direction of at least one of the ends.

28. The device of claim 27 wherein the cross-sectional area of at least one of the n flexible segments increases linearly in at least one dimension.

29. The device of claim 27 wherein the cross-sectional area of at least one of the n flexible segments increases progressively in at least one dimension.

30. The device of claim 1 wherein the longitudinal carrier is disposed relative to the bone anchoring devices such that the maximum dimension of the cross-sectional surface of at lease one of the n flexible segments, disposed orthogonally to the longitudinal axis, is disposed transversely to the central axes of the bone anchoring devices.

31. The device of claim 1 wherein the longitudinal carrier further comprises two stiff segments and at least one flexible segment there between, each stiff segment connected to a respective bone anchoring device via the connecting part, the two stiff segments each being at least twice as stiff in bending as the at least one flexible segment.

32. A device for the dynamic stabilization of bones or bone fragments comprising:
a longitudinal carrier having a longitudinal axis and comprising in the axial direction:
a number of n flexible segments where n is ≧1, each having a cross-sectional surface orthogonal to the longitudinal axis that corresponds neither to a circle nor to an equilateral polygon, the modulus of elasticity of the material of at least one of the flexible segments changing along the longitudinal axis,
a number of m substantially stiff segments where m=(n+1), and
a total number z of flexible and stiff segments where z is ≧(2n+1); and
at least two bone anchoring devices each having a connecting part and an anchoring part that attaches to bone, each bone anchoring device detachably fastenable to the longitudinal carrier via the connecting part.

33. A device for the dynamic stabilization of bones or bone fragments comprising:
a longitudinal carrier having a longitudinal axis and comprising in the axial direction:
a number of n flexible segments where n is ≧1, each having a cross-sectional surface orthogonal to the longitudinal axis that corresponds neither to a circle nor to an equilateral polygon,
a number of m substantially stiff segments where m=(n+1), at least one of the n flexible segments is connectable to an adjoining stiff segment, and
a total number z of flexible and stiff segments where z is ≧(2n+1); and
at least two bone anchoring devices each having a connecting part and an anchoring part that attaches to bone, each bone anchoring device detachably fastenable to the longitudinal carrier via the connecting part.
